(19) United States
(12) Patent Application Publication
(10) Pub. No.: US 2026/0026721 A1
(43) Pub. Date: Jan. 29, 2026

(54) CIRCULATING TUMOR CELLS COLLECTION AND THERAPY SYSTEM

(71) Applicant: LIFE THERAPEUTICS LTD., Or Yehuda (IL)

(72) Inventors: Adrian Paz, Petah Tikva (IL); Yehuda Bachar, Yehud-Monosson (IL)

(73) Assignee: LIFE THERAPEUTICS LTD., Or Yehuda (IL)

(21) Appl. No.: 19/099,053

(22) PCT Filed: Aug. 9, 2023
(86) PCT No.: PCT/IL2023/050833

§ 371 Date: Jan. 28, 2025

Related U.S. Application Data
(60) Provisional application No. 63/397,820, filed on Aug. 13, 2022.

(51) Int. Cl. A61B 5/1473; A61B 5/145; A61B 5/15; A61B 5/157; A61M 1/34; A61M 1/36; B03C 1/01; B03C 1/03; B03C 1/033

(52) U.S. Cl. CPC A61B 5/14735; A61B 5/14546; A61B 5/150992; A61B 5/157; B03C 1/01; B03C 1/03; B03C 1/0332; A61M 1/34; A61M 1/3618; B03C 2201/18

(57) ABSTRACT

The present invention in some embodiments thereof relates to an apheresis device for collecting and/or removing substances from the blood, such as, circulating tumor cells (CTCs), selected proteins, lipids, pathogens, various harmful substances, etc. More particularly, but not exclusively, the device may be used to reduce the amount of CTCs and/or for personalized immunotherapy.

### RELATED APPLICATION/S

This application is a continuation in part of Internation Application no. PCT/IL2023/050833 filed 9 Aug. 2023 which claims the benefit of priority under 35 USC § 119(e) of U.S. Provisional Patent Application No. 63/397,820 filed 13 Aug. 2022. The contents of Internation Application no. PCT/IL2023/050833 filed 9 Aug. 2023 and U.S. Provisional Patent Application No. 63/397,820 filed 13 Aug. 2022 are incorporated herein by reference in their entirety. This application claims the benefit of priority of Internation Application no. PCT/IL2023/050833 filed 9 Aug. 2023 and U.S. Provisional Patent Application No. 63/397,820 filed 13 Aug. 2022.

### FIELD AND BACKGROUND OF THE INVENTION

The present invention in some embodiments thereof relates to an apheresis device for collecting circulating tumor cells (CTCs) and, more particularly, but not exclusively, to reduce CTCs and/or for personalized immunotherapy.

Circulating Tumor Cells (CTCs) are cancer cells that split away from the primary tumor and appear in the circulatory system as singular units or clusters. CTCs migrate and implantation occurs at a new site (such as the lungs, brain, and bones), in a process commonly known as tumor metastasis.

While survival rates have increased significantly over the past few decades because of progress made in radiology and tissue biopsy, making early detection and diagnosis possible. However, liquid biopsy, particularly that involving the collection of CTCs, is a non-invasive method to detect tumor cells in the circulatory system, which can be easily isolated from human plasma, serum, and other body fluids. Compared to traditional tissue biopsies, fluid sample collection has the advantages of being readily available and more acceptable to the patient. Additionally, it can also detect tumor cells in blood earlier and in smaller numbers, possibly allowing for diagnosis prior to any tumor detection using imaging methods.

However, because of the scarcity of CTCs circulating in blood vessels (only a few CTCs among billions of erythrocytes and leukocytes), thorough but accurate detection methods are required for clinical applications, such as to reduce CTCs and/or for personalized immunotherapy.

Bjørk, R and A.R. Insinga appear to disclose in their paper, A topology optimized switchable permanent magnet system, Journal of Magnetism and Magnetic Materials 465 (2018) 106-113 that “The design of a magnetic field source that can switch from a high field to a low field configuration by rotation by 90° of a set of iron pieces is investigated using topology optimization. A Halbach cylinder is considered as the magnetic field source and iron inserts are placed in the air gap of the Halbach cylinder. The ideal shape of these iron inserts is determined as function of the field generated by the Halbach cylinder and as function of the size of the iron segments. The topology optimized structures are parabolic shaped pieces and have a difference in flux density between the high and low positions that is on average 1.29 times higher than optimized regular pole pieces. The maximum increase is a factor of 2.08 times higher than the regular pole pieces.”

Meessen, Koen J., Bart L. J. Gysen, Johannes J. H. Paulides, and Elena A. Lomonov appear to disclose in their article, Three-Dimensional Magnetic Field Modelingof a Cylindrical Halbach Array, in IEEE TRANSACTIONS ON MAGNETICS, VOL. 46, NO. 6, June 2010 that “A semi-analytical description of the 3-D magnetic field distribution of a cylindrical quasi-Halbach permanent magnet array is derived. This model avoids the necessity of time-consuming finite element analyses and allows for fast parameterization to investigate the influence of the number of segments on the magnetic flux density distribution. The segmented magnet is used to approximate an ideal radial magnetized ring in a cylindrical quasi-Halbach array. The model is obtained by solving the Maxwell equations using the magnetic scalar potential and describes the magnetic fields by a Fourier series.”

### SUMMARY OF THE INVENTION

According to an aspect of some embodiments of the invention, there is provided an apheresis device for collecting selected blood components from a subject, including: an outer tube with a blood inlet and a blood outlet; a hollow inner tube configured to reversibly receive a magnetic rod; wherein the apheresis device includes a surface area configured to attract and bind the selected blood components using an electric or magnetic field.

According to some embodiments of the invention, blood of the subject is mixed and reacted with the markers.

According to some embodiments of the invention, the markers include microparticles, nanoparticles, radioisotopes, a photo-acoustic component, nanovesicles, a photodynamic sensitizer, ferromagnetic molecules, ligands, electrostatically charged molecules, or any combination thereof.

According to some embodiments of the invention, the markers are magnetizable, have a magnetic core, or have an electrostatically charged surface.

According to some embodiments of the invention, the surface area includes a plurality of protrusions located within a space between an inner surface of the outer tube and an outer surface of the inner tube.

According to some embodiments of the invention, the plurality of protrusions are radial protrusions extending from the inner surface of the outer tube.

According to some embodiments of the invention, the plurality of protrusions are radial protrusions extending from the outer surface of the inner tube.

According to some embodiments of the invention, the plurality of protrusions are longitudinal protrusions.

According to some embodiments of the invention, the protrusions are made of a ferromagnetic material.

According to some embodiments of the invention, the protrusions are rod shaped.

According to some embodiments of the invention, the surface area is provided with nano or microwires or rods.

According to some embodiments of the invention, the surface area includes a micropatterned section that is micro or nano patterned.

According to some embodiments of the invention, the micropatterned section includes tangential or vertical nanowires, nanorods, nanopillars, or other nano or microstructures.

According to some embodiments of the invention, a charged portion of the surface area is positively charged.

According to some embodiments of the invention, the charged portion results from coating the surface with a cationic natural or synthetic biocompatible polymer.

According to some embodiments of the invention, the charged portion is in electrical contact with one or more electrodes, that are positively charged by a direct current, by intermittent positive electrical pulses, or by positive electrostatic potential.

According to some embodiments of the invention, the positive electrostatic potential ranges between about 1/10 V to about 100 V.

According to some embodiments of the invention, the positive electrode is a foam electrode through which the blood circulates.

According to some embodiments of the invention, the foam electrode includes a conducting polymer, copper, silver, nickel, other metallic alloy, or any combination thereof.

According to some embodiments of the invention, the foam electrode is covered by a polymer layer.

According to some embodiments of the invention, the surface area is coated with cytokines, chemokines, cells, exosomes, DNA, antibodies, RNA, aptamers, chemotherapeutics, small molecules therapeutics, nanoparticles, oncolytic viruses, chemotherapeutics, ligands for markers on selected blood components, slow-release formulations of these components, or a combination thereof.

According to some embodiments of the invention, the inner tube, the outer tube, or the magnetic rod are configured to be rotatable, thereby facilitating separation of the blood components by size and/or electromagnetic properties.

According to some embodiments of the invention, the inner tube or the outer tube is rotatable between a first configuration, wherein an influence of magnetic field facilitates mixing marker particles with blood components and a second configuration wherein a magnetic field is provided for attracting the marker particles with attached blood components to the surface.

According to some embodiments of the invention, the apheresis device further includes an apheresis device, including a mixing element for mixing the blood with a ferromagnetic component and a switchable magnetic field for attracting ferromagnetic particles with attached blood components.

According to some embodiments of the invention, the mixing is performed by switching an orientation of the magnetic fields, leading to the ferromagnetic passing through blood or fluid and linking to blood products to be cleared.

According to some embodiments of the invention, after the ferromagnetic particles are attached to a magnetized surface resulting in clean blood and the cleaned blood is returned to the subject.

According to some embodiments of the invention, the switching is performed using an electromagnet and preferentially a Halbach array or cylinder mechanically changing the orientation of the magnets or distancing the magnets from the surface.

According to some embodiments of the invention, the surfaced is loaded with adhesive components with high avidity for particular blood components.

According to some embodiments of the invention, the surface is rotated within the blood to capture the particular blood components.

According to some embodiments of the invention, the adhesive components are chosen from: antibodies or segments of them, aptamers, peptides, molecular imprinted polymers, nonspecific binders such as lectins, nanoparticles loaded with these elements, and any combination thereof.

According to some embodiments of the invention, the apheresis device further includes a permanent magnetic filter on the blood outlet to capture any escaped markers.

According to some embodiments of the invention, the permanent magnetic filter includes a low gradient magnetic separation or a high gradient magnetic separation containing some porous soft magnetic material.

According to some embodiments of the invention, the apheresis device further includes a first port for introducing an anti-coagulant into a blood flow through the device.

According to some embodiments of the invention, the anti-coagulant is selected the group including: heparin, serine protease inhibitors, direct thrombin inhibitors, citrate, Ethylenediaminetetraacetic acid (EDTA), or any combination thereof.

According to some embodiments of the invention, the apheresis device further includes a second port for introducing an anticoagulant neutralizing agent.

According to some embodiments of the invention, the anticoagulant neutralizing agent is selected from the group consisting of protamine or calcium ion solution.

According to some embodiments of the invention, di-electrophoresis is used to collect blood components from blood of the subject.

According to some embodiments of the invention, the apheresis device further includes a containment chamber.

According to some embodiments of the invention, containment chamber includes a port for sampling, modifying or retrieving the collected blood components.

According to some embodiments of the invention, the blood components are selected from the group including: circulating tumor cells (CTC), proteins, lipids, pathogens, harmful substances or any combination thereof.

According to some embodiments of the invention, the collected circulating tumor cells (CTC) are subjected to electroporation.

According to some embodiments of the invention, the electroporation is low voltage electroporation.

According to some embodiments of the invention, the collected circulating tumor cells (CTC) undergo DNA transfection.

According to some embodiments of the invention, the DNA transfection is assisted by one or more positively charged polymers.

According to some embodiments of the invention, blood of the subject is circulated continuously, or intermittently.

According to an aspect of some embodiments of the invention, there is provided a batch method for use of an apheresis device, the method including: extracting a portion of a subjects' blood from their body; mixing and reacting the blood with markers to selectively bind the markers to selected blood components resulting in a marked blood component; passing the portion of blood with the selected blood component through the apheresis device, wherein the apheresis device is configured to attract specific portions of the markers to a magnetized surface, thereby removing the marked blood components; and returning cleaned blood to a circulatory system of the subject, wherein this method is repeated as many times as required.

According to an aspect of some embodiments of the invention, there is provided a flow through method for use of an apheresis device, the method including: continuously directing blood from a subject directly into an apheresis device; mixing and reacting the blood with markers to selectively bind the markers to selected blood components; attracting specific portions of the markers to a magnetized surface of the apheresis device, thereby removing the selected blood components; and returning cleaned blood to a circulatory system of the subject.

According to some embodiments of the invention, the markers include one or more binding sites for specific blood components.

According to some embodiments of the invention, the markers include one or more magnetic parts configured to facilitate separation of the marked blood component from the blood.

According to some embodiments of the invention, the marker is a microparticle, nanoparticle, radioisotope, photo-acoustic component, nanovesicle, photodynamic sensitizer, ferromagnetic molecule, ligand, electrostatically charged molecule, or any combination thereof.

According to an aspect of some embodiments of the invention, there is provided an adsorption method for use of an apheresis device, the method including: introducing blood of a subject into the apheresis device; attracting and binding selected blood components to a surface of the apheresis device, thereby removing the selected blood components from blood of the subject; and returning cleaned blood to a circulatory system of the subject.

According to some embodiments of the invention, the attracting and binding is to a surface of the apheresis device coated with a selective binder specific to the selected blood component.

According to some embodiments of the invention, the selected blood component is selected from the group including: proteins, lipids, pathogens, harmful substances, CTCs, or any combination thereof.

According to some embodiments of the invention, the method further includes at least one of testing, modifying and neutralizing the blood component after the attracting and binding.

According to some embodiments of the invention, the neutralizing is by electroporation, mechanical removal, cytokines, chemokines, cells, exosomes, DNA, antibodies, RNA, or aptamers, chemotherapeutics, small molecules therapeutics, nanoparticles, or oncolytic viruses, or a combination thereof.

According to some embodiments of the invention, the method further includes returning the neutralized blood component to a circulatory system of the subject.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a schematic illustration of an apheresis device in a peripheral venous catheter, in accordance with an embodiment of the current invention.

FIG. 2 is a schematic illustration of a method for using an apheresis device in accordance with an embodiment of the current invention.

FIG. 3 is a schematic illustration of a wire, in accordance with an embodiment of the current invention.

FIG. 4 is a schematic illustration of an apheresis device in a catheter, in accordance with an embodiment of the current invention.

FIG. 5 is a schematic illustration of an apheresis device, in accordance with an embodiment of the current invention.

FIG. 6 is a schematic illustration of a metallic foam electrode, in accordance with an embodiment of the current invention.

FIG. 7 is a perspective view of a schematic illustration of an apheresis device with radial protrusions, in accordance with an embodiment of the current invention.

FIG. 8 is a cut-away view of a schematic illustration of an apheresis device with radial protrusions, in accordance with an embodiment of the current invention.

FIG. 9 is a cut-away view of a schematic illustration of an apheresis device without protrusions, in accordance with an embodiment of the current invention.

FIG. 10 is a schematic illustration of a magnetic rod in accordance with an embodiment of the current invention.

FIG. 11A and 11B are a perspective view and a cut-away view of a schematic illustration, respectively, of an apheresis device with longitudinal protrusions, in accordance with an embodiment of the current invention.

FIGS. 12A-C are schematic illustrations of an apheresis device in accordance with an embodiment of the current invention.

FIGS. 13A-C are schematic illustrations of an apheresis device in accordance with an embodiment of the current invention.

FIG. 14 is a block diagram of an apheresis device in accordance with an embodiment of the current invention.

FIG. 15 a flow chart for a batch method of use of an apheresis device in accordance with an embodiment of the current invention.

FIG. 16 a flow chart for a flow through method of use of an apheresis device in accordance with an embodiment of the current invention.

FIG. 17 a flow chart for an adsorption method of use of an apheresis device in accordance with an embodiment of the current invention.

### DESCRIPTION OF SPECIFIC EMBODIMENTS OF THE INVENTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The present invention in some embodiments thereof relates to an apheresis device for collecting and/or removing substances from the blood, such as, circulating tumor cells (CTCs), selected proteins, lipids, pathogens, various harmful substances, etc. More particularly, but not exclusively, the device may be used to reduce the amount of CTCs and/or for personalized immunotherapy.

### Overview

Some embodiments relate to a high throughput device for collecting circulating tumor cells (CTC). For example, the throughput may range between 1 to 10 ml/min and/or between 10 to 100 ml/min and/or between 100 to 500 ml/min. According to some embodiments, the high throughput device may be used for collecting blood components such as: circulating cells, exosomes, selected proteins, lipids, pathogens, various harmful substances, or other biological material or molecules. According to some embodiments, the device may provide a large surface area, over which the subjects' blood may be circulated for extended time periods. According to some embodiments, the blood components may be attracted to the surface using an electric and/or magnetic field. Not in the specification, blood is used as an example of fluid that may be treated in an apheresis device. Other fluids may be treated as well. Accordingly, in this specification statements that are made about blood are to be understood as referring to any applicable fluid and can be exchanged with the word fluid. In the claims, the word blood specifically refers to blood, and the word fluid will be used when a broader range of fluids is intended.

According to some embodiments, the device may be a compact apheresis device that operates by drawing blood from the patient and returning it to the patient. Optionally, the apheresis device may reduce CTCs. Optionally, the removed CTCs may be used for generating personalized vaccines, for example, by irreversible electroporation (IRE) of cells in conjunction with relevant adjuvants and/or delivery vehicles for personalized cancer vaccine and/or immunotherapy.

According to some embodiments, the device may be an apheresis device. As used herein, the term “apheresis” may relate to a process in which a particular substance or part of someone's blood is removed from the blood and the rest returned to the body, e.g., separation into plasma and cells, the reintroduction of the cells, used especially to remove antibodies in treating autoimmune diseases, etc.

According to some embodiments, the apheresis device may be implantable. Optionally, the device may be implanted within the body of a subject which may bypass and/or may be incorporated into part of the subjects' circulatory system, e.g., a vein, artery, lymphatic vessel. Optionally, the apheresis device may include two or more connectors for attaching to a subjects' circulatory system. Optionally, the apheresis device may include a switch for diverting the blood flow.

According to some embodiments, the apheresis device may be an external device. Optionally, the external apheresis device may include two or more connectors for attaching to a subjects' circulatory system, e.g., cannula, catheter, etc. According to some embodiments, the apheresis device may be portable and/or wearable. According to some embodiments, the apheresis device may be a stent through which blood may be circulated. Optionally, the blood may be circulated in the apheresis device for about 10 min to about 3 hours, and/or about 3 hours to about 24 hours, and/or about 1 day to about 6 days, and/or about 7 days to about 6 months. Optionally, the apheresis device may include a switch for diverting the blood flow.

According to some embodiments, the apheresis device may include and/or may be connected to a collection and/or containment chamber. Optionally, the collected material may be collected in the collection and/or containment chamber. Optionally, the collected material in the collection and/or containment chamber may be sampled, and/or modified and/or removed and/or returned to the subject. Optionally, “cleaned” blood from which circulating cells, exosomes, selected proteins, lipids, pathogens, various harmful substances, or other biological material or molecules have been removed may be returned to the subject. Optionally, the apheresis device may include one or more ports to sample and/or remove the collected material.

According to some embodiments, the flow rate of the blood circulating through the apheresis device may be between about 1 ml/min to about 10 ml/min, and/or between about 10 ml/min to about 100 ml/min, and/or between about 100 ml/min to about 300 ml/min. Optionally, the apheresis device may include and/or may be attached to a peristaltic pump for circulating the blood.

According to some embodiments, the apheresis device may be used in a batch method and/or a flow through method. Optionally, the subjects' blood may be circulated continuously, and/or intermittently through the apheresis device.

In some embodiments, a magnetic array may be in the form of a Halbach array. Optionally, the magnetic array may have multiple configurations. For example, the array may include parts that move with respect to each other. For example, an array of magnets may move with respect to a second array of magnets and/or elements of contrasting magnetic permeability (for example ferric elements). For example, moving elements in a magnetic array may increase, decrease and/or reorient a magnetic field. For example, there may be an outer circular magnet surrounding the outer tube. The outer magnet may be organized as a circular Halbach array. The Halbach cylinder may be axially or rotationally movable. A soft magnetic material such as iron alloy may be positioned on part of the outer tube circumference. Rotation of the Halbach cylinder in relation to these soft magnetic materials will concentrate the magnetic field lines in some configuration reducing or eliminating the magnetic field within the container for example as illustrated in image 4 from Bjørk, R and A.R. Insinga appear to disclose in their paper, A topology optimized switchable permanent magnet system, Journal of Magnetism and Magnetic Materials 465 (2018) 106-113.)

In some embodiments, a tube may be provided with a helical means that mix the blood during rotation of the inner tube. For example, a helical stirrer and/or magnet may be rotate in a tube and/or blood may be passed through a helical tube of resulting in desired mixing and/or centrifugal forces. Optionally the helix may have constant and/or varying diameter and/or may not be circular.in cross section.

In some embodiments, the system may include a single tube and/or multiple tubes. In case of multiple tube, the tube, the tubes may be concentrically and/or eccentrically oriented.

In some embodiments, there may be one or more permanent magnetic filter on the blood outflow to capture any escaped magnetic nanoparticles. Such magnetic filter may be a low gradient magnetic separation or a high gradient magnetic separation containing some porous soft magnetic material.

According to some embodiments, a marker may selectively bind to a target molecule (e.g., protein, lipid, exosomes, various harmful substances, etc.) and/or a target cell, pathogen, etc. Optionally, the marker may aid in selective collection of a target molecule and/or cell. Optionally, the apheresis device may include a surface coated with a marker. Optionally, the apheresis device may include one or more ports trough which a marker molecule may be added to the blood. Optionally, a marker may be injected into a circulatory system of a subject upstream to the apheresis device.

According to some embodiments, the marker may be a microparticle, nanoparticle, radioisotope, photo-acoustic component, nanovesicle, photodynamic sensitizer, marker molecule (e.g., ferromagnetic molecule, ligand, or electrostatically charged molecule), or any combination thereof. Optionally, the marker may be magnetizable or magnetic core, or have an electrostatically charged surface.

According to some embodiments, the marker may be mixed with the afferent blood flow. Optionally, the marker may be linked to the blood components. Optionally, marked blood components may be attracted by the corresponding electrical and/or magnetic field in the apheresis device and collected, e.g., onto the large surface area of the apheresis device and/or in an associated collection and/or containment chamber. Optionally, the marked molecules and/or marked cells may be reversibly adsorbed onto one or more surfaces of the apheresis device.

Some embodiments relate to a method for collecting and/or neutralizing circulating tumor cells (CTC) using the apheresis device. Optionally, the neutralizing may be performed using electroporation, and/or mechanical removal, and/or subjecting the cells to chemotherapeutic agents, and/or oncolytic viruses (e.g., attached to the large surface area of the apheresis device) and/or a combination thereof.

Some embodiments relate to a method for active immunization using the apheresis device. Optionally, the collected tumor cells may be used for whole cell immunization by electroporating the tumor cells, and/or exposing and/or transfecting the cells within the apheresis device to different substances, such as: cytokines, chemokines, cells, exosomes, DNA, antibodies, RNA, aptamers, chemotherapeutics, small molecules therapeutics, nanoparticles, oncolytic viruses, and/or a combination thereof.

Some embodiments relate to a method for clearing the blood of selected blood components and/or toxic substances using the apheresis device.

Some embodiments relate to a method for treating malignant diseases, chronic inflammatory diseases or auto-immune diseases using the apheresis device. Optionally, immunologic cells with a specific phenotype may be collected. Optionally, the collected immunologic cells may be loaded and/or transfected with different substances, such as: cytokines, chemokines, exosomes, DNA, antibodies, RNA, aptamers, chemotherapeutics, small molecules therapeutics, nanoparticles, oncolytic viruses, and/or a combination thereof for treating the disease.

According to some embodiments, the collected immunologic cells may have surface receptors for homing to a particular tissue. Optionally, the collected immunologic cells may have particular surface molecules which may facilitate loading target molecules onto and/or into the cells, or a particular DNA sequence, or RNA sequence. Optionally, the target may facilitate transfection of specific homing receptors into the cells. In a particular embodiment, the loading or transfection may be performed by any known method or device.

According to some embodiments, the apheresis device may include a surface exposed to the blood flow. Optionally, the surface may have a large surface area. Optionally, the large surface area may be provided by a plurality of protrusions. Optionally, the plurality of protrusions may be radial rods and/or longitudinal rods. Optionally, the plurality of protrusions may be configured to capture and/or release marked cells and/or marked molecules. Optionally, the plurality of protrusions may be charged. Optionally, the plurality of protrusions may be configured to produce an electric and/or magnetic field. Optionally, the plurality of protrusions may be coated with a substance to attract and/or capture marked cells and/or marked molecules.

According to some embodiments, the plurality of protrusions may be radial rods mounted on a tube of the apheresis device. Optionally, the plurality of protrusions may be mounted on an inner surface of an outer tube of the apheresis device (e.g., on the inner walls of the apheresis device). Optionally, the plurality of protrusions may be mounted on an outer surface of an inner tube encapsulated by an outer tube of the apheresis device. Optionally, the plurality of protrusions may be located on an inner surface of an outer tube of the apheresis device and/or an outer surface of an inner tube encapsulated by an outer tube of the apheresis device. Optionally, the radial rods may be nanowires. Optionally, the nanowires may be composed of and/or coated with a metal. Optionally, the apheresis device may not include a plurality of protrusions.

According to some embodiments, the plurality of protrusions may be longitudinal rods. Optionally, the longitudinal rods may extend along all and/or part of the length of the apheresis device. Optionally, the longitudinal protrusions may protrude into the space between an inner surface of the outer tube and an outer surface of the inner tube. Optionally, the longitudinal rods may be nanowires and/or may be coated with nanowires. Optionally, the nanowires may be composed of and/or coated with a metal. Optionally, the apheresis device may not include a plurality of protrusions.

According to some embodiments, one or more components of the apheresis device may be rotatable. Optionally, the outer tube may be rotatable. Optionally, the inner tube may be rotatable. Optionally, a magnetic rod inserted into a hollow of the inner tube may be rotatable. Optionally, the speed and/or direction of rotation may be controllable. Optionally, rotation may facilitate separation of various blood components by size and/or electromagnetic properties.

According to some embodiments, the device may be on a catheter, or wire made structure that is inserted into a vein, and/or an artery, and/or lymphatic vessel.

According to some embodiments, the apheresis device may include a wire. Optionally, the wire may be plated with a metal. Optionally, the wire may include a plurality of vertical nanowires and/or protrusions. Optionally, the wire may be of a helical shape of constant or variable radius. Optionally, the distance between the loops of the wire coil may be in the range between about 0.1 mm to about 5 mm, and/or between about 0.5 mm to about 3 mm, and/or between about 1 mm to about 1.5 mm. Optionally, the wire diameter may be in the range between about 0.1 mm to about 5 mm, and/or between about 0.5 mm to about 3 mm, and/or between about 1 mm to about 1.5 mm. For example, the apheresis device may include nanowires wrapped around a thin copper wire.

According to some embodiments, the apheresis device may include a port for introducing an anti-coagulant, such as, but not limited to heparin, serine protease inhibitors, direct thrombin inhibitors, citrate, Ethylenediaminetetraacetic acid (EDTA), etc. Optionally, the port may be located in the vicinity of and/or at the blood flow inlet. Optionally, an anti-coagulant may be inserted into the blood flow upstream of the apheresis device.

According to some embodiments, the apheresis device may include a port for introducing an anticoagulant neutralizing agent, such as: protamine or calcium ion solution. Optionally, the port may be located in the vicinity of and/or at the blood flow outlet. Optionally, an anti-coagulant neutralizing agent may be inserted into the blood flow downstream of the apheresis device.

According to some embodiments, the apheresis device may include a first port for introducing an anti-coagulant and/or the apheresis device may include a second port for introducing an anticoagulant neutralizing agent.

According to some embodiments, the apheresis device may include a port for sampling and/or retrieving the collected cells, and/or for washing the cells, and/or for adding different substances to the blood such as: cytokines, chemokines, cells, exosomes, DNA, antibodies, RNA, calcium or sodium, compounds, aptamers, chemotherapeutics, small molecules therapeutics, nanoparticles, oncolytic viruses and/or a combination thereof.

According to some embodiments, the collected cells may be subjected to electroporation. Optionally, the electroporation may be low voltage electroporation. Optionally, the electroporation may use nanowires and/or patterned microelectrodes. Optionally, the collected tumor cells may be used for whole cell immunization by electroporating the tumor cells and/or, exposing or transfecting the cells within the device to different substances such as: cytokines, chemokines, cells, exosomes, DNA, antibodies, RNA, aptamers, chemotherapeutics, small molecules therapeutics, nanoparticles, oncolytic viruses and/or a combination thereof. Optionally, DNA transfection may be assisted by positively charged polymers, such as, but not limited to: polyethylenimine (PEI), poly-lysine, etc.

According to some embodiments, a surface of the apheresis device may be positively charged (e.g., one or more of a plurality of protrusions and/or a surface of the inner and/or outer tubes). Optionally, the positive charge may result from coating the surface with a cationic natural and/or synthetic biocompatible polymer, such as, but not limited to: cationic cellulose, cationic dextran, cationic dextrin, chitosan, polyethylenimine (PEI), poly(l-lysine) (PLL), polyamidoamine (PAA), poly(amino-co-ester) (PAEs), poly[2-(N,N-dimethylamino)ethyl methacrylate] (PDMAEMA), etc.

According to some embodiments, a surface area of the apheresis device (e.g., a plurality of protrusions) may be in contact with one or more electrodes, Optionally, the electrodes may be positively charged by a direct current, and/or by intermittent positive electrical pulses, and/or by pure positive electrostatic potential. Optionally, the positive potential may range between about 0.1 V to about 100 V, and/or between about 1 V to about 50 V, and/or between about 10 V to about 25 V.

According to some embodiments, the electrode surfaces may be covered by nanowires. According to some embodiments, the electrodes may be a foam electrode through which the blood circulates. Optionally, the nanowires may be constructed from a metallic foam and/or metal coated foam. Optionally, the foam electrodes may comprise a metal, such a copper, silver, nickel, others metallic alloy, and/or a combination thereof. Optionally, the foam electrode may comprise an electrically conductive metal and/or conductive polymer. Optionally, the foam electrode may be covered by a polymer layer. Optionally, the use of foam electrodes may increase the surface area of the apheresis device.

According to some embodiments, a surface area of the apheresis device may be micro or nano patterned to increase its surface area. Optionally, such patterning may be performed by providing tangential and/or vertical nanowires, and/or nanorods, and/or nanopillars, and/or other nano or microstructures.

According to some embodiments, a surface area of the apheresis device may be coated with one or more anti-coagulants, such as, but not limited to: heparin, NO-catalytic bioactivity coating, albumin, C1-esterase inhibitor (C1-INH) coating, phosphorylcholine (PC), poly(2-methoxy-ethyl-acrylate) (PMEA), polyethylene oxide (PEO), poly(MPC-co-BMA-co-TSMA) (PMBT), endothelial coating, and/or any combination thereof to prevent blood clotting within the device,

According to some embodiments, a surface area of the apheresis device may be coated with one or more antibodies, aptamers, and/or other ligands. Optionally, such molecules may be markers for the CTC. Optionally, the ligands may be for markers of epithelial and/or mesenchymal differentiation, and/or specific cell lineage and/or tumor markers.

According to some embodiments, a surface area of the apheresis device may be coated with one or more cytokines, chemokines, pattern recognition molecules (PAMP), vaccines' adjuvants, cells, exosomes, DNA, or RNA encoding these proteins or antibodies or surface markers, aptamers, chemotherapeutics, such as, but not limited to: bleomycin, cisplatin, small molecules therapeutics, nanoparticles, oncolytic viruses, calcium compounds, slow-release formulations of these components, and/or a combination thereof.

According to some embodiments, a surface area of the apheresis device may be coated with one or more theragnostic products, such as, but not limited to: nanoparticles, radioisotopes, photo-acoustic components, nanovesicles, photodynamic sensitizers, ferromagnetic nanoparticles, etc. Optionally, such theragnostic products may be used for detection using PET, MRI, CT, ultrasound, etc. Optionally, electrical impedance and/or di-electrophoresis spectroscopy, and/or electromagnetic waves spectroscopy may be used to determine cell types and/or count. For example, an electric field for separating blood components may use an electrical field having a voltage potential of between 100 to 300 V and/or between 300 to 1000 V and/or between 1000 to 2000 V and/or between 2000 to 5000 V.

According to some embodiments, di-electrophoresis may be used to collect cells from the apheresis device.

Some embodiments relate to a batch method of separation of marked molecules and/or marked cells from a subjects' blood. For example, the method may include removing a portion of a subjects' blood from their body, mixing and/or reacting the blood with marker molecules (e.g., nanoparticles, etc.) to selectively bind the markers to selected cells and/or blood components (e.g., proteins, lipids, etc.). Optionally, the marker molecule may include one or more binding sites for particular molecules and/or cells. Optionally, the marker molecule may include one or more magnetic parts configured to facilitate separation of the marked cell and/or marked molecule. The marked blood may then be passed through an apheresis device. Optionally, the apheresis device may be configured to attract specific portions of the marker molecule to a magnetized surface. Optionally, the cleaned blood may be returned to the subjects' circulatory system. Optionally, this process may be repeated with a subsequent batch of blood from the subject. Optionally, the removed marked cells and/or marked molecules may be tested, and/or modify and/or selected/separated cells and/or molecules (e.g., modified CTC's) may be returned to the subjects' circulatory system.

Some embodiments relate to a flow through method of separation of marked molecules and/or marked cells from a subjects' blood. For example, blood from a subject may be directed directly into an apheresis device. In the apheresis device, mixing and/or reacting the blood with marker molecules (e.g., nanoparticles, etc.) to selectively bind the markers to selected cells and/or blood components (e.g., proteins, lipids, etc.). Optionally, the marker molecule may include one or more binding sites for particular molecules and/or cells. Optionally, the marker molecule may include one or more magnetic parts configured to facilitate separation of the marked cell and/or marked molecule. Optionally, the apheresis device may be configured to attract specific portions of the marker molecule to a magnetized surface. Optionally, the cleaned blood may be returned to the subjects' circulatory system. Optionally, the removed marked cells and/or marked molecules may be tested, and/or modify and/or selected/separated cells and/or molecules (e.g., modified CTC's) may be returned to the subjects' circulatory system.

Some embodiments relate to adsorption of molecules and/or cells from a subjects' blood. For example, a subjects' blood may be introduced into an apheresis device in a batch method and/or a flow through method (i.e., intermittently and/or continuously). Various blood components (e.g., cells, proteins, lipids, pathogens, harmful substances, CTCs, etc.) may be attracted to a surface of the apheresis device coated with a selective binder for specific to a selected blood component. Optionally, the selective binder may be magnetic. Optionally, the cleaned blood may be returned to the subjects' circulatory system. Optionally, the removed molecules and/or cells may be collected in a collection and/or containment chamber. Optionally, this process may be continuous. Optionally, the removed cells and/or molecules may be collected and subsequently tested, and/or modify and/or selected/separated cells and/or molecules (e.g., modified CTC's) may be returned to the subjects' circulatory system. Optionally, some or all of the removed material may be destroyed during the collection process.

Some embodiments may relate to treatment of a subject by inactivation of CTCs. Optionally, the CTCs collected by the apheresis device may be inactivated (e.g., by electroporation, chemically, transfection of RNA and/or DNA, etc.). Optionally, the inactivated CTCs may be reintroduced to the circulatory system of the subject. Optionally, the inactivated CTCs may be used as a vaccine to activate immune system against CTCs. Optionally, an active substance may be added to the CTCs (or other kinds of cells) such that the modified CTCs may collect on the growth and/or tissue to be treated, and the active substance may kill and/or treat the growth and/or affected tissue. Optionally, the active substance may be a chemotherapeutic substance, radioactive substance, or combination thereof. In some embodiments, modifications may include DNA or RNA, or other biotherapeutics or markers transfection. For example, an electric field for electroporation may use an electrical field having a voltage potential of between 100 to 300 V and/or between 300 to 1000 V and/or between 1000 to 2000 V and/or between 2000 to 5000 V.

### Specific Embodiments

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

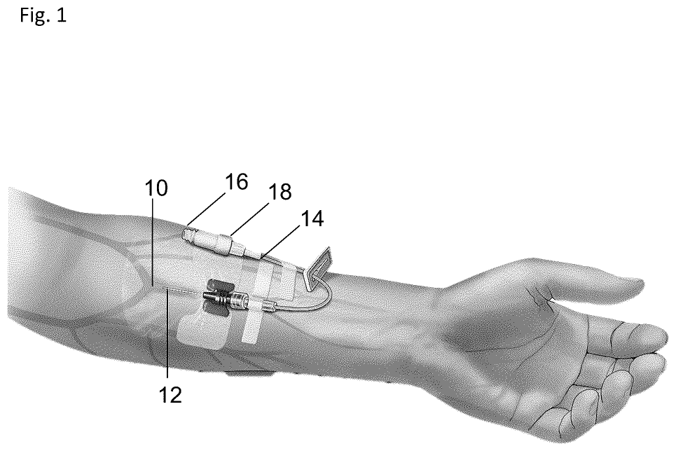

FIG. 1 is a schematic illustration of an apheresis device in a peripheral venous catheter, in accordance with an embodiment of the current invention. For example, the apheresis device 18 may include and/or may be connected to a catheter 12, or wire made structure that is inserted into a vein 10, an artery or lymphatic vessel. The subjects' blood flows into the apheresis device 18 through a flow inlet 14 and out of the apheresis device 18 through a flow outlet 16, where it is returned to a vein 10, an artery or lymphatic vessel of the subject. Optionally, the device may have a stent like structure.

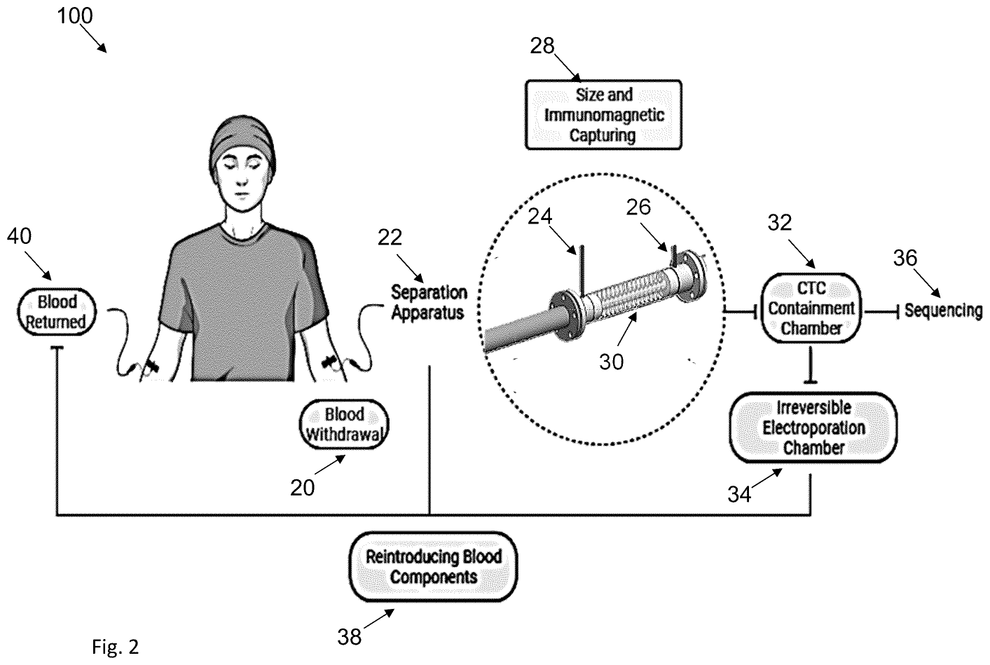

FIG. 2 is a schematic illustration of a method for using an apheresis device in accordance with an embodiment of the current invention. For example, in method 100, in a first step, a subject is connected to the apheresis device using a catheter line e.g., a peripheral or central venous catheter line. The connection may last between 1 to 10 minutes and/or between 10 to 30 minutes and/or between 30 mins to about 3 hrs., during which CTCs are collect from the blood. Optionally, the procedure can be carried out at a clinic or at home, with supervision from a caretaker, if needed. In a second step, blood is withdrawn 20 from the subject and introduced into the apheresis device 30 and fed into the separation apparatus 22 through a blood inlet 24 where it is separated into components and/or processed. Optionally, the workflow may include processes of enrichment, filtration, washing, characterization, and imaging (e.g., automated fluorescence) of the cell population and antigens. Blood flows through an outlet 26 of the device back to the subject and/or to other processes.

According to some embodiments, processing may include one or more of the following:
1. Enrichment: The blood may be introduced into the separation apparatus 22 in rapid flow. For example, the inner tube/core of the apheresis device may be rotated. By controlling fluid flow, and/or rotation speed cells experience varying forces depending on their size. These forces may determine whether a cell is captured 18 and/or flushed. Optionally, cells that attach non-specifically may be flushed by fluid drag forces.
2. Immunomagnetic capture 28: Functionalized immunomagnetic beads or surfaces may contact cells and specifically bind CTCs. Immunofuncinalization of the beads and surfaces may include tumor-associated antigens such as EpCAM, HER/new2, PSMA, Cytokeratins, Vimentin, N-cadherin, PD-L1, MART1, CEA, MUC-1, folic acid receptors or any other cell capturing antibody. Optionally, beads may be functionalized with adjuvants delivery for downstream applications.
3. Magnetic separation of CTCs from blood: a magnetic rod may be introduced into the core to apply a strong magnetic field and magnetically capture selected cells. Optionally, the separation apparatus may then be primed for further use.
4. CTC processing: a magnetic rod may be removed from the hollow core. Separated cells may then be washed into a containment chamber 32 for further processing, e.g., sequencing 36, electroporation 34, etc.
5. Electroporation chamber 34: CTCs may be subjected to controlled irreversible electroporation (IRE), for example, to generate immunologic cell death.
6. Reintroduction 38 and/or cancer vaccination: IRE cell products may be coronated with adjuvants carried on the nanoparticles and/or introduced downstream and reintroduced 38 to the subjects' blood stream 40. Optionally, captured cells may be expanded in-vitro to sufficient numbers for cancer vaccination (e.g., personalized immunotherapy). Optionally, using specially designed scaffolds, such as cryogels (for example, made of natural polymers such as, chitosan, alginate and/or synthetic polymers, such as polyethylene glycol, polyvinyl alcohol). In some embodiments, the reintroduced material may create a local immune-stimulating environment.

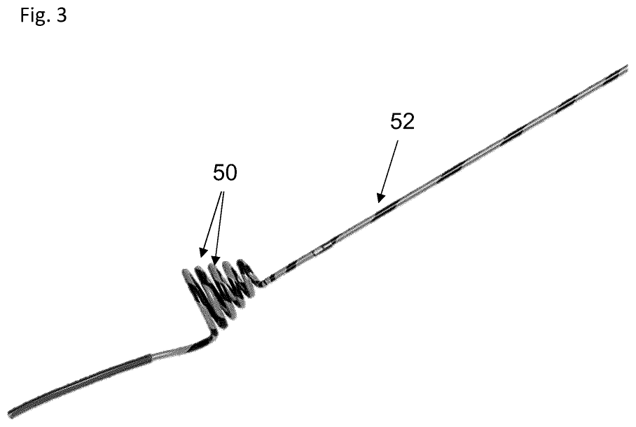

FIG. 3 is a schematic illustration of a wire in accordance with an embodiment of the current invention. For example, the wire may be of a helical shape of constant or variable radius. Optionally, the distance between the loops of the wire coil 50 may be in the range between about 0.1 mm to about 5 mm, and/or between about 0.5 mm to about 3 mm, and/or between about 1 mm to about 1.5 mm. Optionally, the wire diameter 52 may be in the range between about 0.1 mm to about 5 mm, and/or between about 0.5 mm to about 3 mm, and/or between about 1 mm to about 1.5 mm.

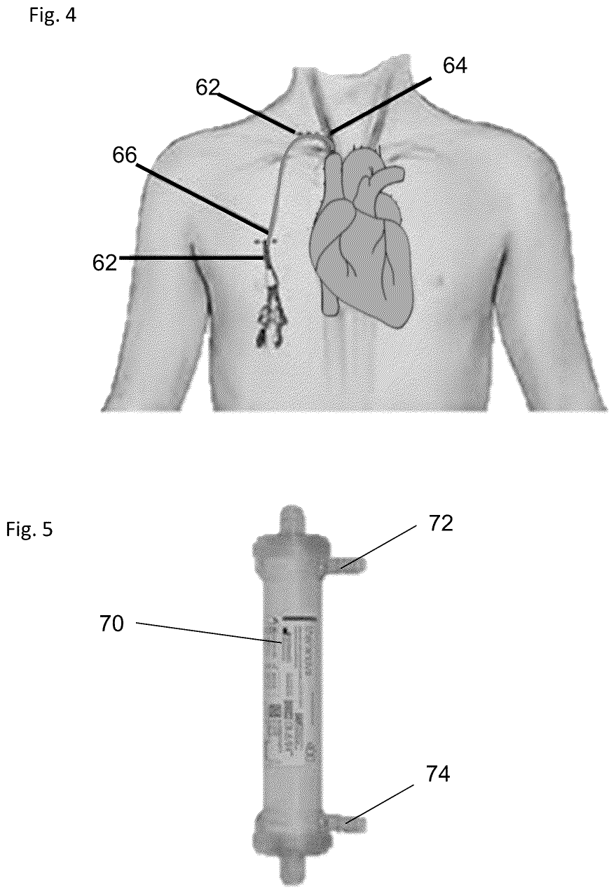

FIG. 4 is a schematic illustration of an apheresis device in a catheter, in accordance with an embodiment of the current invention. For example, the stent may include a wire which may be inserted at an insertion site 62 into a vein 64 via a catheter, which may come out of the chest. Optionally, the catheter may be kept in place by a cuff 66. Optionally, vertical nanowires may be included on the stent surface.

According to some embodiments, the apheresis device may include a port for introducing an anti-coagulant, such as, but not limited to heparin, serine protease inhibitors, direct thrombin inhibitors, citrate, Ethylenediaminetetraacetic acid (EDTA), etc. Optionally, the port may be located in the vicinity of and/or at the blood flow inlet.

According to some embodiments, the apheresis device may include a port for introducing an anticoagulant neutralizing agent, such as: protamine or calcium ion solution. Optionally, the port may be located in the vicinity of and/or at the blood flow outlet.

According to some embodiments, the apheresis device may include a first port for introducing an anti-coagulant and/or the apheresis device may include a second port for introducing an anticoagulant neutralizing agent.

According to some embodiments, the apheresis device may include a switch for diverting the blood flow.

According to some embodiments, the apheresis device may include a port for sampling and/or retrieving the collected cells, and/or for washing the cells, and/or for adding different substances to the blood such as: cytokines, chemokines, cells, exosomes, DNA, antibodies, RNA, calcium or sodium, compounds, aptamers, chemotherapeutics, small molecules therapeutics, nanoparticles, oncolytic viruses and/or a combination thereof.

FIG. 5 is a schematic illustration of an apheresis device, in accordance with an embodiment of the current invention. For example, the subjects' blood flows into the apheresis device 70 through a flow inlet 72 and out of the apheresis device 70 through a flow outlet 74, where it is returned to a vein, an artery or lymphatic vessel of the subject. Optionally, the subjects' blood may be circulated continuously, or intermittently over a surface within an apheresis device.

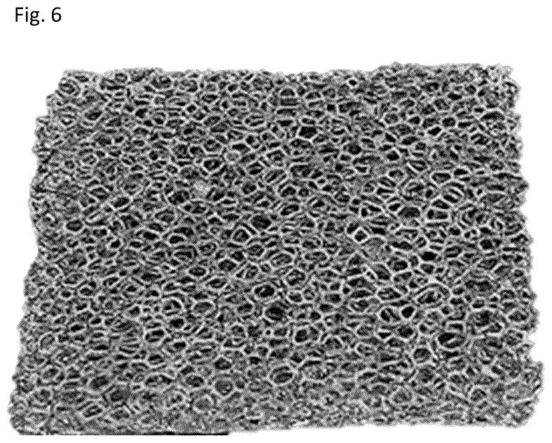

FIG. 6 is a schematic illustration of a metallic foam electrode, in accordance with an embodiment of the current invention. For example, the positive electrode may be a foam electrode through which the blood circulates. Optionally, the foam electrode may be made of a material, such copper, and/or silver, and/or nickel, and/or others metallic alloy, and/or a conducting polymer. Optionally, the positive electrode may comprise a multitude of channels.

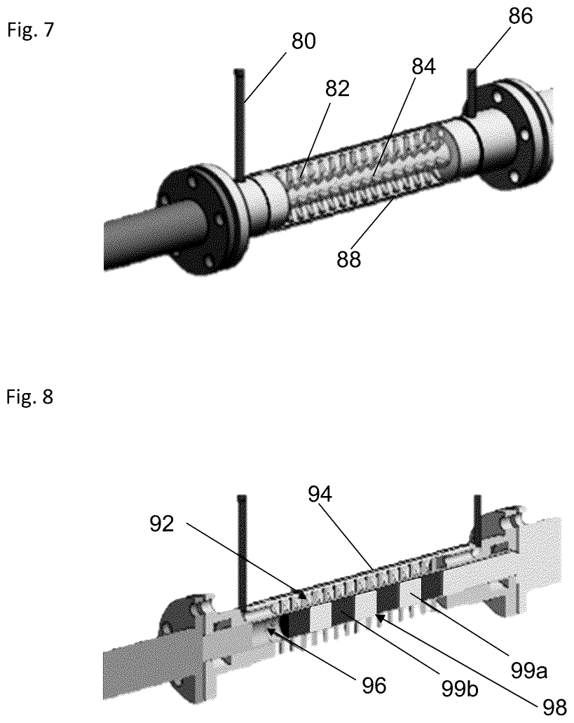

FIG. 7 is a perspective view of a schematic illustration of an apheresis device with radial protrusions, in accordance with an embodiment of the current invention. For example, the apheresis device may be constructed from an outer tube 88. Optionally, the outer tube 88 may have a length ranging between about 3 cm to about 5 cm, and/or between about 5 cm to about 7 cm, and/or between about 7 cm to about 10 cm. Optionally, the tube may have a diameter ranging between about 1 cm to about 2 cm, and/or between about 2 cm to about 3 cm, and/or between about 1.5 cm to about 2.5 cm. Optionally, the tube 88 may encapsulate a metallic hollow core/tube 84 with radial protrusions 82 (for example, there may be 10 to 100 protrusions and/or between 100 to 1000 protrusions and/or between 1000 to 10000 protrusions). Optionally, the radial protrusions 82 may be bare, and/or coated with immunocapturing moieties. Optionally, the subjects' blood flows into the apheresis device through a flow inlet 80 and out of the apheresis device through a flow outlet 86.

According to some embodiments, the plurality of protrusions may be radial rods mounted on a tube of the apheresis device. Optionally, the plurality of protrusions may be mounted on an inner surface of an outer tube of the apheresis device (e.g., on the inner walls of the apheresis device). Optionally, the plurality of protrusions may be mounted on an outer surface of an inner tube encapsulated by an outer tube of the apheresis device. Optionally, the plurality of protrusions may be located on an inner surface of an outer tube of the apheresis device and/or an outer surface of an inner tube encapsulated by an outer tube of the apheresis device. Optionally, the radial rods may be nanowires. Optionally, the nanowires may be composed of and/or coated with a metal. Optionally, the apheresis device may not include a plurality of protrusions.

FIG. 8 is a cut-away view of a schematic illustration of an apheresis device with radial protrusions, in accordance with an embodiment of the current invention. For example, the apheresis device may include an outer tube 94 and may encapsulate a metallic hollow inner tube 96 with several hundreds to thousands of protrusions 92. Optionally, the plurality of protrusions 92 may be radial protrusions. Optionally, magnetic rod 98 may be inserted and/or withdrawn reversibly from inner tube 96. Optionally, magnetic rod 98 may be designed to induce a strong magnetic field gradient to rapidly capture magnetically labeled CTCs. Optionally, the protrusions may be nanowires and/or patterned microelectrodes. In some embodiments the magnetic rod 98 includes sections with opposing polarity. For example, sections 99a, 99b of opposing polarities may be arranged and/or vary longitudinally and/or circumferentially. The arrangement of the section 99a, 99b along and/or around the rod 98 may determine the polarity of protrusion 92. For example, sliding the rod 98 and/or rotating the rod 98 inside the inner tube 96 may cause temporal changes in the polarity of the protrusions. Optionally, different polarity of sections 99a, 99b along the rod 98 may result in different polarities of different protrusions 92.

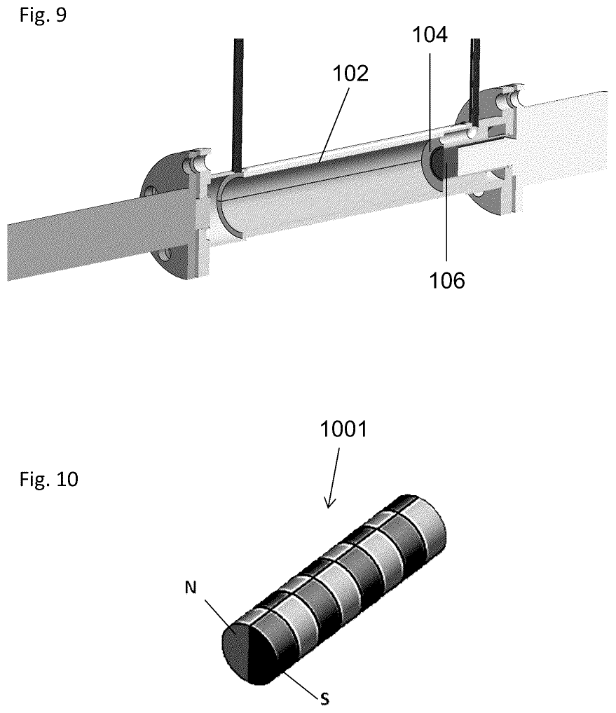

FIG. 9 is a cut-away view of a schematic illustration of an apheresis device without protrusions, in accordance with an embodiment of the current invention. For example, the apheresis device may not include a plurality of protrusions. Optionally, the apheresis device may include an outer tube 102 and may encapsulate a metallic hollow inner tube 104. Optionally, the outer tube, the inner tube or both may rotate. Optionally, the rotating may be at a controlled and/or calculated rate to enrich the medium with CTC and/or to facilitate magnetic nanoparticle capture. Optionally, by controlling fluid flow through the apheresis device, and/or rotation speed of inner tube, cells and/or blood components may experience varying forces depending on their size, electrostatic charge, polarity, and or attachment to a particle. Optionally, cells that attach non-specifically may be flushed by fluid drag forces.

Optionally, the core of the hollow inner tube 104 may include a magnetic rod 106. Optionally, the magnetic rod 106 may be reversibly inserted and withdrawn from the core of the hollow inner tube.

According to some embodiments, during the enrichment and/or immunomagnetic capture, the core may spin at a controlled and/or calculated rate enriching the medium with CTC and facilitating magnetic nanoparticle capture. Optionally, during the magnetic separation phase, a magnetic rod may be mechanically introduced into the hollow core. Optionally, the magnetic rod may be designed to induce a strong magnetic field gradient to rapidly capture magnetically labeled CTCs.

FIG. 10 is a schematic illustration of a magnetic rod 1001 in accordance with an embodiment of the current invention. For example, the magnetic rod 1001 may include one or more bands (e.g., light bands with a N polarity and/or black bands having S polarity), components or sections with poles of alternating polarity and/or an alternating current may be applied to the magnetic rod 1001 to generate a strong magnetic field. The rod 1001 may be moved longitudinally and/or rotated e.g., to create alternating magnetic field.

In some embodiments, a magnetic rod 1001 may be introduced into the core of the apheresis device to apply a strong magnetic field and/or magnetically capture selected cells, blood components, etc. Optionally, the magnetic rod may be removed from the hollow core to prime the apheresis device for further use. In some embodiments, a magnetic rod may be moved and/or rotated to cause variation in the field for example, the change the polarity of molecules and/or protrusions and/or cause magnetic attraction or repulsion.

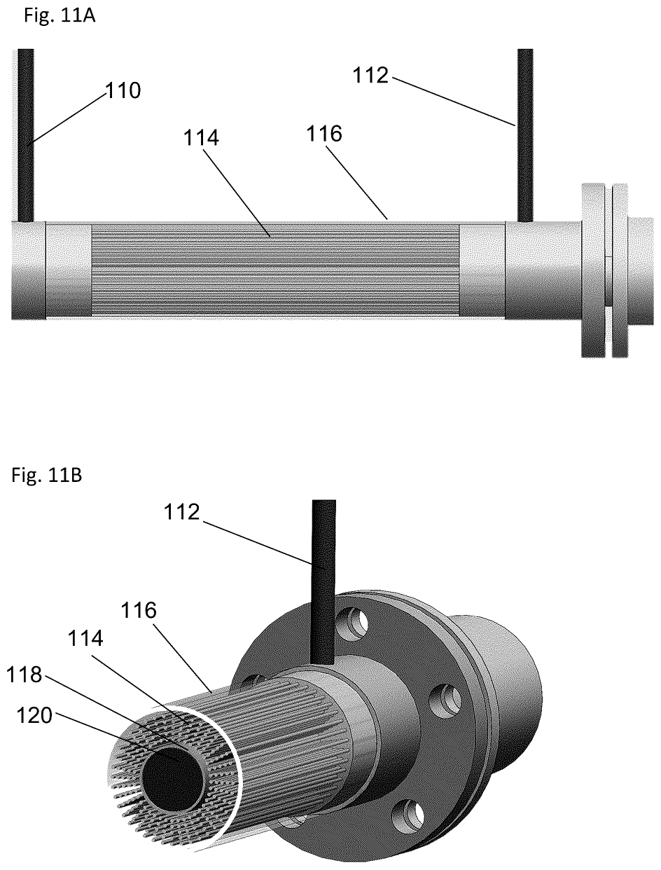

FIGS. 11A and 11B are a perspective view and a cut-away view of a schematic illustration, respectively, of an apheresis device with longitudinal protrusions, in accordance with an embodiment of the current invention. For example, the subjects' blood flows into the apheresis device through a flow inlet 110 and out of the apheresis device through a flow outlet 112 in the outer tube 116. The subjects' blood may flow over several hundreds to thousands of longitudinal protrusions 114 protruding into the space between an encapsulated metallic hollow inner tube 118 and the outer tube 116. A magnetic rod 120 may be reversibly inserted into the hollow of the inner tube 118 to produce a strong magnetic field to magnetically capture selected cells, blood components, etc.

According to some embodiments, the plurality of protrusions may be longitudinal rods. Optionally, the longitudinal rods may extend along all and/or part of the length of the apheresis device. Optionally, the longitudinal protrusions may protrude into the space between an inner surface of the outer tube and an outer surface of the inner tube. Optionally, the longitudinal rods may be nanowires and/or may be coated with nanowires. Optionally, the nanowires may be composed of and/or coated with a metal. Optionally, the apheresis device may not include a plurality of protrusions.

According to some embodiments, the protrusions may be positively charged. Optionally, the positive charge may result from coating the surface of the protrusions with a cationic natural and/or synthetic biocompatible polymer, such as, but not limited to: cationic cellulose, cationic dextran, cationic dextrin, chitosan, polyethylenimine (PEI), poly(l-lysine) (PLL), polyamidoamine (PAA), poly(amino-co-ester) (PAEs), poly[2-(N,N-dimethylamino)ethyl methacrylate] (PDMAEMA), etc.

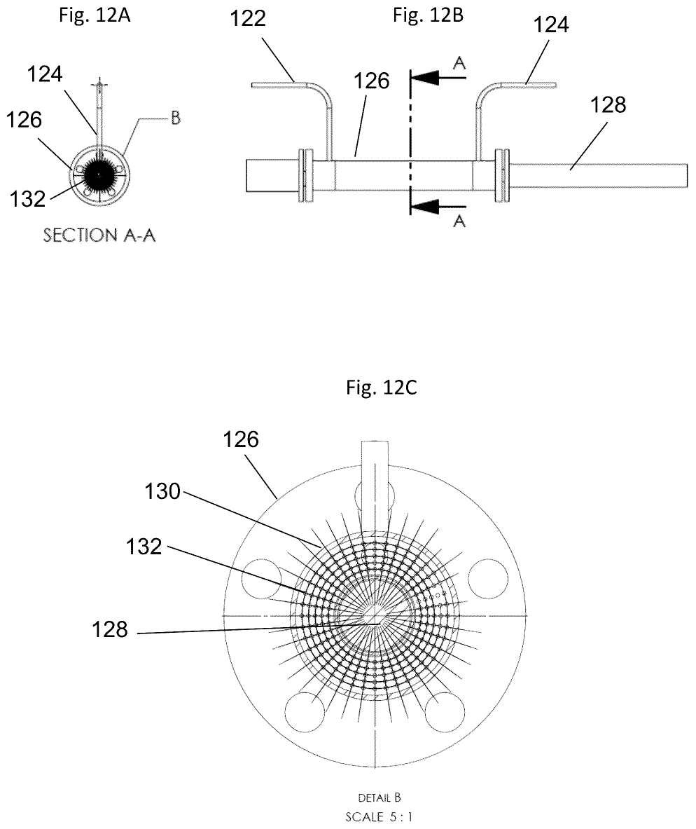

FIGS. 12A-C are schematic illustrations of an apheresis device in accordance with an embodiment of the current invention. For example, the subjects' blood flows into the apheresis device through a flow inlet 122 and out of the apheresis device through a flow outlet 124 in the outer tube 126. The subjects' blood may flow over several hundreds to thousands of protrusions 132 which may be radial and/or longitudinal. A magnetic rod 128 may be reversibly inserted into the hollow of the inner tube 130 to produce a strong magnetic field to magnetically capture selected cells, blood components, etc.

According to some embodiments, the protrusions may be coated with one or more anti-coagulants, such as, but not limited to: heparin, NO-catalytic bioactivity coating, albumin, C1-esterase inhibitor (C1-INH) coating, phosphorylcholine (PC), poly(2-methoxy-ethyl-acrylate) (PMEA), polyethylene oxide (PEO), poly(MPC-co-BMA-co-TSMA) (PMBT), endothelial coating, and/or any combination thereof to prevent blood clotting,

According to some embodiments, the protrusions may be coated with one or more antibodies, aptamers, and/or other ligands which may be markers on the CTC. Optionally, the ligands may be for markers of epithelial and/or mesenchymal differentiation, and/or specific cell lineage and/or tumor markers.

According to some embodiments, the protrusions may be coated with one or more cytokines, chemokines, pattern recognition molecules (PAMP), vaccines' adjuvants, cells, exosomes, DNA, or RNA encoding these proteins or antibodies or surface markers, aptamers, chemotherapeutics, such as, but not limited to: bleomycin, cisplatin, small molecules therapeutics, nanoparticles, oncolytic viruses, calcium compounds, slow-release formulations of these components, and/or a combination thereof.

According to some embodiments, the protrusions may be coated with one or more theragnostic products, such as, but not limited to: nanoparticles, radioisotopes, photo-acoustic components, nanovesicles, photodynamic sensitizers, ferromagnetic nanoparticles, etc. Optionally, such theragnostic products may be used for detection using PET, MRI, CT, ultrasound, etc. Optionally, electrical impedance and/or di-electrophoresis spectroscopy, and/or electromagnetic waves spectroscopy may be used to determine cell types and/or count.

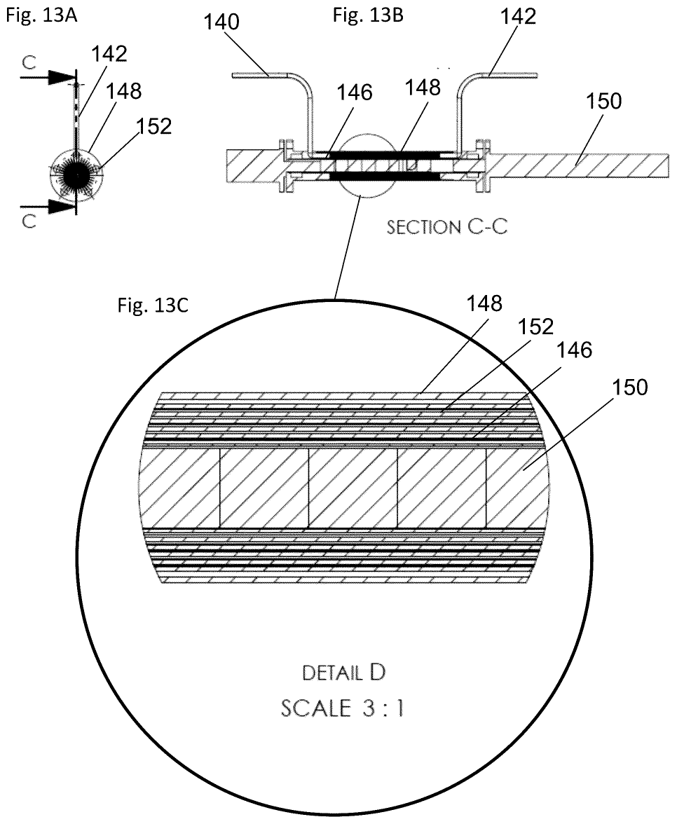

FIGS. 13A-C are schematic illustrations of an apheresis device in accordance with an embodiment of the current invention. For example, the subjects' blood flows into the apheresis device through a flow inlet 140 and out of the apheresis device through a flow outlet 142 in the outer tube 148. The subjects' blood may flow over several hundreds to thousands of longitudinal protrusions 152.

A magnet may reversibly magnetize the protrusions. For example, the magnetic rod may be inserted into the hollow 150 of the inner tube 146 and/or contact one or both longitudinal ends of the protrusions. Optionally, the magnet may magnetize the longitudinal protrusions and/or. produce a strong magnetic field to magnetically capture selected blood components (e.g., cells, lipids, proteins, etc.). Optionally, the protrusions may have various lengths.

According to some embodiments, the protrusions may be in electrical contact with one or more electrodes. For example, the electrodes may run through the inner tube and/or contact the protrusions (e.g., one or both longitudinal ends of the protrusions). Optionally, the electrodes may be positively charged by a direct current, and/or by intermittent positive electrical pulses, and/or by pure positive electrostatic potential. Optionally, the positive potential may range between about 0.1 V to about 100 V, and/or between about 1 V to about 50 V, and/or between about 10 V to about 25 V. Optionally, the electrode surfaces may be covered by nanowires. Optionally, the electrodes may be a foam electrode through which the blood circulates. Optionally, the foam electrodes may comprise a metal, such a copper, silver, nickel, others metallic alloy, and/or a combination thereof. Optionally, the foam electrode may comprise an electrically conductive metal and/or conductive polymer. Optionally, the foam electrode may be covered by a polymer layer. Optionally, surfaces may be covered by nanowires.

According to some embodiments, the protrusions may be micro or nano patterned to increase its surface area. Optionally, such patterning may be performed by providing tangential and/or vertical nanowires, and/or nanorods, and/or nanopillars, and/or other nano or microstructures.

According to some embodiments, nano or microparticles, may be associated with cells and/or selected blood components and/or ligands, for binding to the blood components. Optionally, the nano or microparticles may have specific electrostatic charges. Optionally, the particles may have a magnetizable or magnetic core, or have an electrostatically charged surface. Optionally, the nano or microparticles may be mixed with the afferent blood flow. Optionally, the particles may be linked to the blood components. Optionally, linked particles may be attracted by the corresponding electrical and/or magnetic field and collected on the large surface area (protrusions) of the apheresis device.

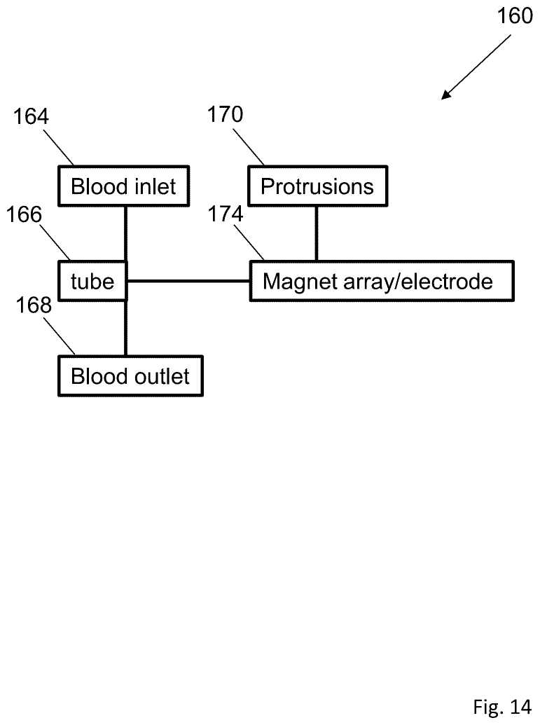

FIG. 14 is a block diagram of an apheresis device in accordance with an embodiment of the current invention. For example, the subjects' blood flows into the apheresis device 160 through a flow inlet 164 and out of the apheresis device 160 through a flow outlet 168 in the tube 166. Optionally, the subjects' blood may flow over several hundreds to thousands of radial and/or longitudinal protrusions 170 protruding into the space in the tube. In some embodiments, there may be an inner tube inside tube 166. A magnet and/or electrode 174 in the form of rod may be reversibly inserted into the hollow of the inner tube to produce a strong magnetic field to magnetically capture selected cells, blood components, etc. and then removed to allow them to mix and/or free them. Alternatively or additionally, the magnet array 174 may be reconfigurable. For example, the array may include a Halbach array. For example, the array may be moved and/or rotated to switch between a configuration where a strong side of the array faces the fluid and/or where a weak side faces the fluid. Alternatively or additionally, the relationship between two sub-arrays may be switched to strengthen and/or weaken the field (e.g., for capturing and/or freeing particles). For example, the sub-arrays may include concentric rings of magnets and/or concentric rods where adjacent magnets may be rotated to strengthen one another in one configuration and/or to interfere with each other in a different configuration and/or there may be a magnetic transmissive material that is moved with relation to the array to direct/strengthen/weaken the field in different configurations and/or there may be non-concentric arrays of magnets that are moved with relation to one another to switch configurations. Alternatively or additionally, there may be an electromagnet whose direction and/or strength may be changed. Alternatively or additionally, attraction and/or repulsion may be electrostatic based on electrodes that change in strength or polarity. Switching the magnetic and/or electric fields may switch the system configuration. For example, in one configuration the system may collect markers and/or blood components. For example, in a second configuration the system may release markers and/or blood components. For example, in another configuration the system may mix markers and/or blood components.

In some embodiments, the magnet may be contacted to the protrusions and/or an electrical current may be used to produce a magnetic field in or around the protrusions (e.g., the coil may be integrated into the protrusions and/or wrap around the protrusions). In some embodiment, an electrode may be used for example to establish an electrical charge on the protrusions and/or cause adhesion and/or repulsion. Optionally, the magnetic field and/or electrical charge may be alternated, for example, by moving the magnet/electrode 174 and/or changing its polarity.

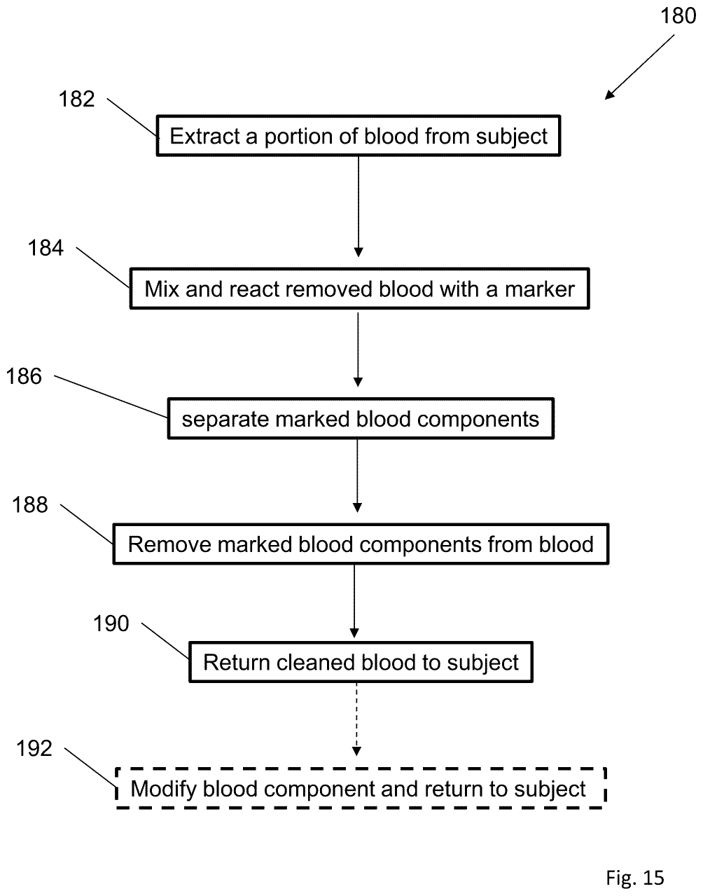

FIG. 15 a flow chart for a batch method of use of an apheresis device in accordance with an embodiment of the current invention. For example, in method 180, a portion of a subjects' blood is extracted 182 from their body, mixing 184 and/or reacting the blood with markers (e.g., nanoparticles, etc.) to selectively bind the markers to selected blood components (e.g., CTCs, proteins, lipids, etc.). Optionally, the markers may include one or more binding sites for particular molecules and/or cells. Optionally, the markers may include one or more magnetic parts configured to facilitate separation of the marked blood components.

In some embodiments, the marked blood components may then be separated 186. For example, separation may be performed while the blood passes through an apheresis device. Optionally, the apheresis device may be configured to attract specific portions of the marker to a magnetized surface, thereby removing 188 the marked blood components. Optionally, the cleaned blood may be returned 190 to the subjects' circulatory system. Optionally, this process may be repeated with a subsequent batch of blood from the subject. Optionally, the removed marked blood components may be tested, and/or modify 192 and/or selected/separated cells and/or molecules (e.g., modified CTC's) may be returned to the subjects' circulatory system.

In some embodiments, the mixing 184 markers and blood may be performed in the same chamber as separation 186 and/or removal 188 of the desired blood component. For example, the chamber may include a magnetic array configured to be reconfigured. For example, the array may include parts that move with respect to each other. For example, an array of magnets may move with respect to a second array of magnets and/or elements of contrasting magnetic permeability (for example ferric elements). For example, moving elements in a magnetic array may increase, decrease and/or reorient a magnetic field. For example, there may be an outer circular magnet surrounding the outer tube. The outer magnet may be organized as a circular Halbach array. The Halbach cylinder may be axially or rotationally movable. A soft magnetic material such as iron alloy may be positioned on part of the outer tube circumference. Rotation of the Halbach cylinder in relation to these soft magnetic materials will concentrate the magnetic field lines in some configuration reducing or eliminating the magnetic field within the container for example as illustrated in image 4 from Bjørk, R and A.R. Insinga appear to disclose in their paper, A topology optimized switchable permanent magnet system, Journal of Magnetism and Magnetic Materials 465 (2018) 106-113.) Alternatively or additionally, attraction and/or repulsion of particles may be related to electrostatic forces and/or the chamber may be changed from a mixing chamber to a separation chamber by changing polarity and/or charge of electrodes. Alternatively or additionally, attraction and/or repulsion of particles may include electromagnets and/or the chamber may be changed from a mixing chamber to a separation chamber by changing direction and/or magnitude of current in the electromagnet.

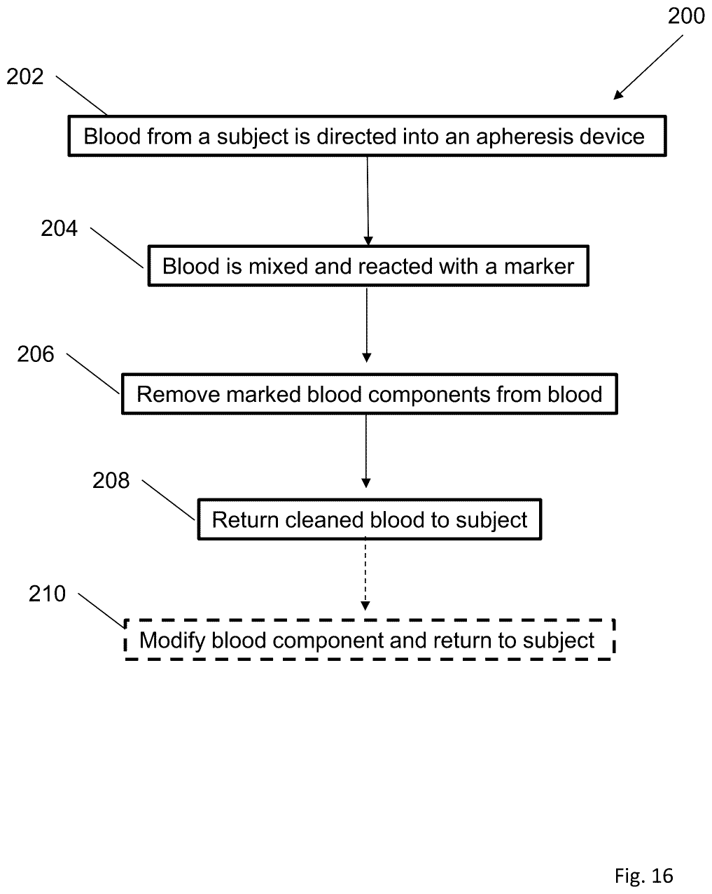

FIG. 16 a flow chart for a flow through method of use of an apheresis device in accordance with an embodiment of the current invention. For example, in method 200, blood from a subject may be directed 202 directly into an apheresis device. In the apheresis device, mixing 204 and/or reacting the blood with markers (e.g., nanoparticles, etc.) to selectively bind the markers to selected blood components (e.g., CTCs, proteins, lipids, etc.). Optionally, the markers may include one or more binding sites for particular blood components. Optionally, the markers may include one or more magnetic parts and/or charges parts configured to facilitate separation of the marked blood components. Optionally, the apheresis device may be configured to attract specific portions of the marker molecule to a magnetized and/or charged surface, thereby removing 206 the marked blood components.

In some embodiments, the blood is mixed 204 with markers in the same chamber where the removing 206 occurs. For example, during a mixing stage, an electric and/or magnetic separator may be disabled (e.g., disconnecting an electrode, disconnecting an electromagnet, removing a magnet, rearranging magnetic elements to weaken the field (for example, by interference and/or changing a configuration of a magnetic array (e.g., a Halbach Array)).

In some embodiments, the cleaned blood may be returned 208 to the subjects' circulatory system. Optionally, the removed marked blood components may be collected in a collection and/or containment chamber. Optionally, this process may be continuous. Optionally, the removed marked blood components may be tested, and/or modify and/or selected/separated cells and/or molecules (e.g., modified CTCs) may be returned 210 to the subjects' circulatory system.

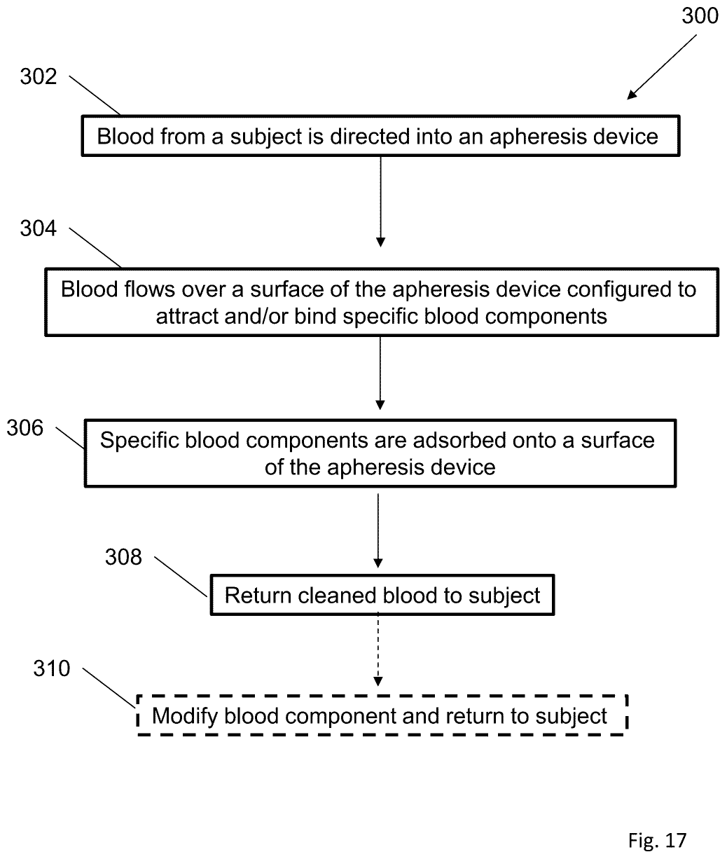

FIG. 17 a flow chart for an adsorption method of use of an apheresis device in accordance with an embodiment of the current invention. For example, in method 300, a subjects' blood may be introduced 302 into an apheresis device in a batch method and/or a flow through method (i.e., intermittently and/or continuously). The blood may flow 304 over a surface configured to attract and/or bind to specific blood components. Various blood components (e.g., cells, proteins, lipids, pathogens, harmful substances, CTCs, etc.) may be adsorbed 306 onto a surface of the apheresis device coated with a selective binder for specific to a selected blood component. Optionally, the selective binder may be magnetic and/or electrically charged. In some embodiments, a surface of the device may be magnetized and/or electrically charged continuously and/or at different times e.g., to attract or repel particles. Optionally mixing and/or moving surface may be used to increase contact and/or speed up activities. For example, an attraction and reaction surface may be rotated inside the medium. Optionally, the cleaned blood may be returned 308 to the subjects' circulatory system. Optionally, the removed cells and/or molecules may be collected and subsequently tested, and/or modified and/or selected/separated cells and/or molecules (e.g., modified CTC's) may be returned 310 to the subjects' circulatory system. Optionally, some or all of the removed material may be destroyed during the collection process.

It is expected that during the life of a patent maturing from this application many relevant technologies will be developed, and the scope of the terms are intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In the claims:

1. An apheresis device for collecting selected blood components from a subject, comprising:
an outer tube with a blood inlet and a blood outlet;
a hollow inner tube configured to reversibly receive a magnetic rod; wherein the apheresis device includes a surface area configured to attract and bind the selected blood components using an electric or magnetic field.

2. The apheresis device of claim 1, including a plurality of markers and wherein blood of the subject is mixed and reacted with the markers.

3-4. (canceled)

5. The apheresis device of claim 1, wherein the surface area comprises a plurality of protrusions located within a space between an inner surface of the outer tube and an outer surface of the inner tube.

6-10. (canceled)

11. The apheresis device of claim 5, wherein the surface area is provided with nano or microwires or rods.

12-13. (canceled)

14. The apheresis device of claim 1, wherein a charged portion of the surface area is positively charged.

15-16. (canceled)

17. The apheresis device of claim 14, wherein a positive electrostatic potential of said charged portion ranges between 1/10 V to 100 V.

18. The apheresis device of claim 1, further including a foam electrode through which the blood circulates.

19-21. (canceled)

22. The apheresis device of claim 1, wherein the inner tube, the outer tube, or the magnetic rod are configured to be rotatable, thereby facilitating separation of the blood components by size and/or electromagnetic properties.

23. The apheresis device of claim 22, wherein the inner tube or the outer tube is rotatable between a first configuration, wherein an influence of magnetic field facilitates mixing marker particles with blood components and a second configuration wherein a magnetic field is provided for attracting the marker particles with attached blood components to the surface.

24. The apheresis device of claim 1, further comprising: a mixing element for mixing the blood with a ferromagnetic component and a switchable magnetic field for attracting ferromagnetic particles with attached blood components.

25. The apheresis device of claim 24, wherein the mixing is performed by switching an orientation of the magnetic fields, leading to the ferromagnetic passing through blood or fluid and linking to blood products to be cleared.

26. (canceled)

27. The apheresis device of claim 25, wherein said switching is performed using an electromagnet and preferentially a Halbach array or cylinder mechanically changing the orientation of the magnets or distancing the magnets from the surface.

28. The apheresis device of claim 1, wherein said surfaced is loaded with adhesive components with high avidity for particular blood components and the surface is rotated within the blood to capture the particular blood components.

29-40. (canceled)

41. The apheresis device of claim 1, wherein the blood component includes circulating tumor cells (CTC) and the CTC's are subjected to electroporation.

42. The apheresis device of claim 41, wherein the electroporation is low voltage electroporation.

43. The apheresis device of claim 1, wherein the blood component includes circulating tumor cells (CTC) and the CTC's undergo DNA or RNA, or other biotherapeutics or markers transfection.

44-49. (canceled)

50. A flow through method for use of an apheresis device, the method comprising: continuously directing blood from a subject directly into an apheresis device; moving or rotating element releasable connected to ferromagnetic markers to selectively bind the ferromagnetic markers to selected blood components;
returning cleaned blood to a circulatory system of the subject.

51. The method of claim 50, wherein the markers include one or more binding sites for specific blood components and one or more magnetic parts configured to facilitate separation of the marked blood component from the blood.

52-54. (canceled)

55. An adsorption method for use of an apheresis device, the method comprising: introducing blood of a subject into the apheresis device;
attracting and binding selected blood components to a movable or surface of a rotation element of the apheresis device, thereby removing the selected blood components from blood of the subject; and
returning cleaned blood to a circulatory system of the subject.

56. The method of claim 55, wherein said attracting and binding is to a surface of the apheresis device coated with a selective binder specific to the selected blood component.

57-60. (canceled)
